(19) United States
(12) Patent Application Publication
(10) Pub. No.: US 2014/0088586 A1
(43) Pub. Date: Mar. 27, 2014

(54) RENAL NERVE MODULATION DEVICES

(71) Applicant: BOSTON SCIENTIFIC SCIMED, INC., Maple Grove, MN (US)

(72) Inventors: KIM G. DAVIS, MINNEAPOLIS, MN (US); DEREK C. SUTERMEISTER, HAM LAKE, MN (US); CASS A. HANSON, ST. PAUL, MN (US); THOMAS P. JANCARIC, MAPLE GROVE, MN (US)

(73) Assignee: BOSTON SCIENTIFIC SCIMED, INC., MAPLE GROVE, MN (US)

(21) Appl. No.: 14/038,374

(22) Filed: Sep. 26, 2013

Related U.S. Application Data
(60) Provisional application No. 61/705,948, filed on Sep. 26, 2012.

(51) Int. Cl. A61B 18/18

(52) U.S. Cl. CPC A61B 18/18
USPC 606/33

(57) ABSTRACT

Medical devices and methods for making and using medical devices are disclosed. An example medical device may include a renal nerve modulation device. The renal nerve modulation device may include an elongate shaft. A balloon may be coupled to the shaft. The balloon may have a hydrophilic electrode region. A flexible electrode may be coupled to the shaft and may be disposed within the balloon. The flexible electrode may have a plurality of openings formed therein.

### CROSS-REFERENCE TO RELATED APPLICATIONS

This application claims priority under 35 U.S.C. §119 to U.S. Provisional Application Ser. No. 61/705,948, filed Sep. 26, 2012, the entirety of which is incorporated herein by reference.

### TECHNICAL FIELD

The present disclosure pertains to medical devices and methods for making and using medical devices. More particularly, the present disclosure pertains to medical devices for renal nerve modulation.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a renal nerve modulation device. The renal nerve modulation device may include an elongate shaft. A balloon may be coupled to the shaft. The balloon may have a hydrophilic electrode region. A flexible electrode may be coupled to the shaft and may be disposed within the balloon. The flexible electrode may have a plurality of openings formed therein.

Another example renal nerve modulation device may include an elongate shaft. A balloon may be coupled to the shaft. The balloon may include an inner layer and an outer layer. A hydrophilic electrode region may be defined in the balloon by the absence of the outer layer along a portion of the balloon. A tubular electrode may be disposed about the shaft and may have a plurality of openings formed therein.

An example renal nerve ablation catheter may include an elongate shaft having a distal portion. A tubular electrode may be coupled to the distal portion. The tubular electrode may have a plurality of openings defined therein. A power wire may be attached to the tubular electrode and may extend proximally therefrom. A balloon may be coupled to the shaft. The balloon may have one or more virtual window electrodes disposed thereon.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic view illustrating a renal nerve modulation system in situ;

FIG. 2 is a side view of a portion of an example medical device;

FIG. 3 is an example cross-sectional view taken through line 3-3 in FIG. 2;

FIG. 4 is an example cross-sectional view taken through line 4-4 in FIG. 2;

FIG. 5 is a side view of a portion of another example medical device;

FIG. 6 is a side view of a portion of another example medical device;

FIG. 7 is a side view of a portion of another example medical device; and

FIG. 8 is a side view of a portion of another example medical device.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

Certain treatments require the temporary or permanent interruption or modification of select nerve function. One example treatment is renal nerve ablation, which is sometimes used to treat conditions related to hypertension and/or congestive heart failure. The kidneys produce a sympathetic response to congestive heart failure, which, among other effects, increases the undesired retention of water and/or sodium.

Ablating some of the nerves running to the kidneys may reduce or eliminate this sympathetic function, which may provide a corresponding reduction in the associated undesired symptoms.

While the devices and methods described herein are discussed relative to renal nerve modulation, it is contemplated that the devices and methods may be used in other treatment locations and/or applications where nerve modulation and/or other tissue modulation including heating, activation, blocking, disrupting, or ablation are desired, such as, but not limited to: blood vessels, urinary vessels, or in other tissues via trocar and cannula access. For example, the devices and methods described herein can be applied to hyperplastic tissue ablation, cardiac ablation, pulmonary vein isolation, tumor ablation, benign prostatic hyperplasia therapy, nerve excitation or blocking or ablation, modulation of muscle activity, hyperthermia or other warming of tissues, etc. In some instances, it may be desirable to ablate perivascular renal nerves with ultrasound ablation.

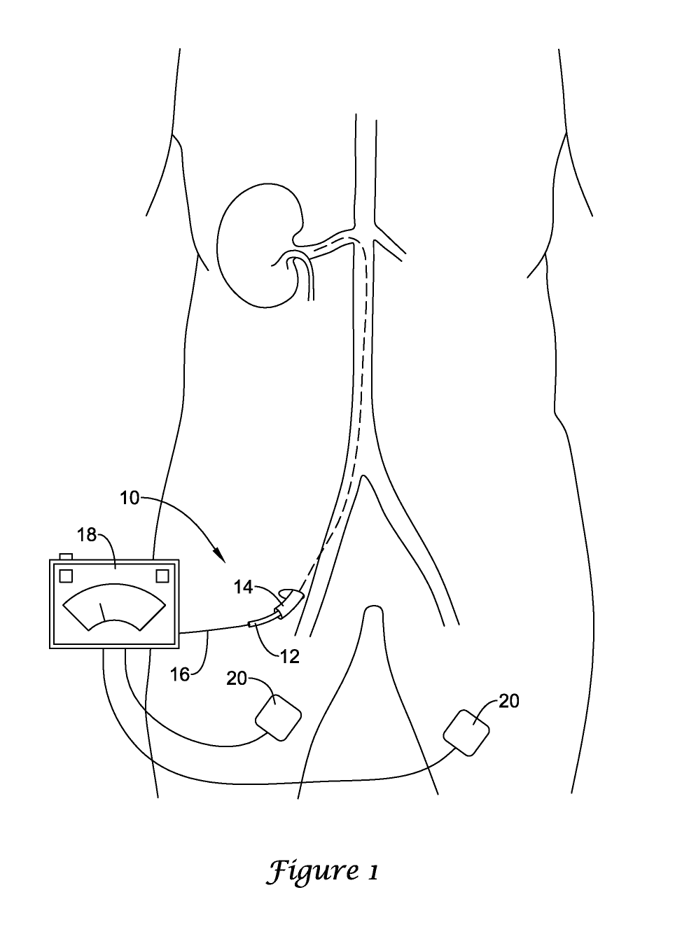

FIG. 1 is a schematic view of an illustrative renal nerve modulation system in situ. System 10 may include one or more conductive element(s) 16 for providing power to a renal ablation system including a renal nerve modulation device 12 and, optionally, within delivery sheath 14, the details of which can be better seen in subsequent figures. A proximal end of conductive element(s) 16 may be connected to a control and power unit 18, which may supply the appropriate electrical energy to activate one or more electrodes disposed at or near a distal end of the renal nerve modulation device 12. In addition, control and power unit 18 may also be utilized to supply/receive the appropriate electrical energy and/or signal to activate one or more sensors disposed at or near a distal end of the renal nerve modulation device 12. When suitably activated, the electrodes are capable of ablating tissue as described below and the sensors may be used to sense desired physical and/or biological parameters. The terms electrode and electrodes may be considered to be equivalent to elements capable of ablating adjacent tissue in the disclosure which follows. In some instances, return electrode patches 20 may be supplied on the legs or at another conventional location on the patient's body to complete the circuit. A proximal hub (not illustrated) having ports for a guidewire, an inflation lumen and a return lumen may also be included.

The control and power unit 18 may include monitoring elements to monitor parameters such as power, voltage, pulse size, temperature, force, contact, pressure, impedance and/or shape and other suitable parameters, with sensors mounted along renal nerve modulation device 12, as well as suitable controls for performing the desired procedure. In some embodiments, the power unit 18 may control a radiofrequency (RF) electrode and, in turn, may “power” other electrodes including so-called “virtual electrodes” described herein. The electrode may be configured to operate at a suitable frequency and generate a suitable signal. It is further contemplated that other ablation devices may be used as desired, for example, but not limited to resistance heating, ultrasound, microwave, and laser devices and these devices may require that power be supplied by the power unit 18 in a different form.

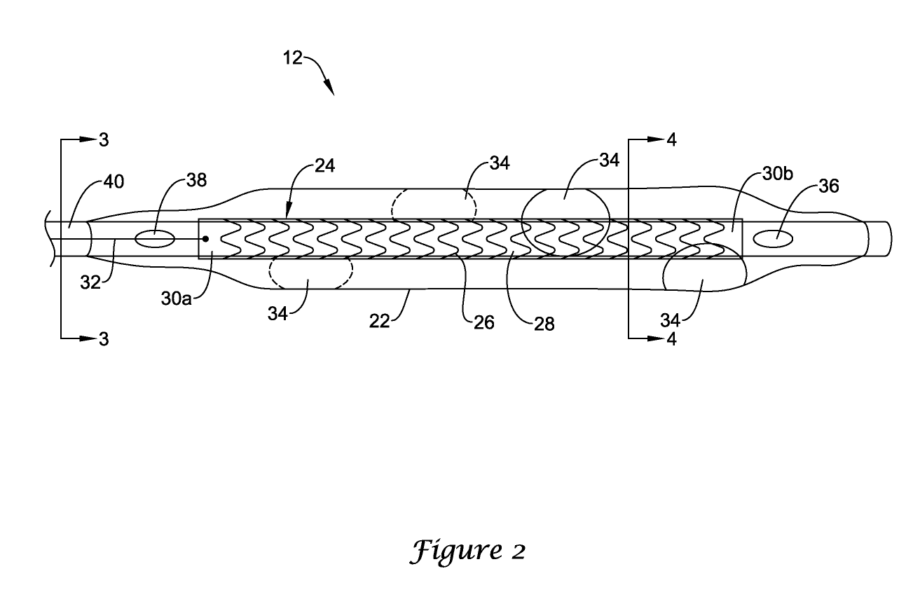

FIG. 2 illustrates a distal portion of a renal nerve modulation device 12. Here it can be seen that renal nerve modulation device 12 may include an elongate member or catheter shaft 40, an expandable member or balloon 22 coupled to shaft 40, and an electrode 24 disposed within balloon 22. When in use, balloon 22 may be filled with a conductive fluid such as saline to allow the ablation energy (e.g., radiofrequency energy) to be transmitted from electrode 24, through the conductive fluid, to one or more windows 34 disposed along or otherwise defined along balloon 22. While saline is one example conductive fluid, other conductive fluids may also be utilized including hypertonic solutions, contrast solution, mixtures of saline or hypertonic saline solutions with contrast solutions, and the like. The conductive fluid may be introduced through a first port or fluid inlet 36 and evacuated through a second port or fluid outlet 38, both in shaft 40. This may allow the fluid to be circulated within balloon 22. The reverse orientation may also be used for ports 36/38 (e.g., where port 36 is a fluid outlet and port 38 is a fluid inlet).

As described in more detail herein, windows 34 may be generally hydrophilic portions of balloon 22 that may absorb fluid (e.g., the conductive fluid) so that energy exposed to the conductive fluid can be conducted to windows 34. Accordingly, windows 34 may take the form of “virtual electrodes” capable of ablating tissue. In some of these and in alternative embodiments, one or more electrodes (not shown) may be attached to an inner or outer surface of balloon 22 and these electrodes may be used to ablate and/or modulate renal nerves.

Electrode 24 may vary in form and/or configuration. In some embodiments, the electrode may include a ribbon that is helically wrapped about shaft 40 and positioned under balloon 22. Such an electrode may be made from, for example, platinum or other suitable materials including those disclosed herein. The use of a platinum ribbon electrode may be effective for renal nerve modulation and/or ablation. However, some power loss may be experienced with these electrodes. For example, when delivering approximately 18 watts of power to the platinum ribbon electrode, approximately 10 watts of power or so may be lost in the form of heat due to the relatively high impedance (resistance) of the electrode. It may be desirable to reduce power loss. It may also be desirable to reduce localized heating or otherwise reduce heating of the electrode in general.

In at least some embodiments, electrode 24 may include a tubular or tube-like electrode having a body portion 26 and a plurality of openings 28 formed therein. In addition to being suitably flexible, such an electrode may have improved power distribution and reduce the impedance (resistance) of the circuit. Consequently, relatively little thermal energy loss may occur along the length of electrode 24. Additionally, only a relatively small temperature gradient may be observed between the proximal and distal ends of electrode 24, indicating that current is able to efficiently dissipate through the conductive fluid to windows 34.

As shown in FIG. 2, electrode 24 may take the form of a stent-like lattice. The precise form and arrangement of the lattice or structure of electrode 24 may vary. For example, a variety of different patterns and/or arrangements may be utilized for electrode 24 including any of those typically utilized for stents. In addition, electrode 24 may also include end caps or pads 30a/30b. Pads 30a/30b may form a convenient location for a power wire 32 (that supplies current) to attach to electrode 24.

In one example, electrode 24 may be a platinum-chromium lattice. The lattice may be about 24 mm in length. Other materials, lengths, and configurations are contemplated including radiopaque materials (which may aid in visualization). Delivery of 18 watts of power to such an electrode 24 may result in a temperature change (relative to the temperature prior to delivery of power) at the proximal end of electrode 24 on the order of about 6.4° C., a temperature change at the middle of electrode 24 on the order of about 4° C., and a temperature change at the distal end of electrode 24 on the order of about 4.4° C. Collectively, this indicates that the latticed electrode 24 may have a temperature drop of only about 2.4° C. along its length, which demonstrates a design having more uniform power distribution along electrode 24. A nickel-titanium alloy lattice electrode 24 also may provide a similar uniform power distribution as evidenced by a relatively low temperature drop along the length of the electrode 24.

Conversely, a 0.003 inch thick, 2 cm platinum ribbon coil electrode may have a temperature change at the proximal end of the electrode on the order of about 16.6° C., a temperature change at the middle of the electrode on the order of about 5.6° C., and a temperature change at the distal end of the electrode on the order of about 2.2° C. This illustrates a more significant temperature drop (e.g., 14.4° C.), indicating a less uniform power distribution. Similarly, a 0.003 inch thick, 2 cm platinum tri-filar coil electrode may have a temperature change at the proximal end of the electrode on the order of about 10.8° C., a temperature change at the middle of the electrode on the order of about 5° C., and a temperature change at the distal end of the electrode on the order of about 3.8° C. (total temperature drop of 7° C.). Moreover, a 0.004 inch thick, 2 cm gold coil electrode may have a temperature change at the proximal end of the electrode on the order of about 14° C., a temperature change at the middle of the electrode on the order of about 4° C., and a temperature change at the distal end of the electrode on the order of about 5.6° C. (total temperature drop of 8.4° C.). Other designs (e.g., a 0.006-0.007 inch silver-copper coil electrodes and/or 0.003 inch gold coil electrodes) also show an increased drop along the length of the electrode. The greater temperature drop along the length of these electrode designs may indicate a less uniform power distribution.

In addition to a more uniform power distribution, a latticed electrode 24 may be desirable for a number of reasons. For example, latticed electrode 24 may be generally more conductive (e.g., by virtue of multiple energy pathways) than a single and/or multi-filar coil electrode. Latticed electrode 24 may also have a reduced circuit impedance, which may allow for increased power delivery to electrode 24, may increase the current density, and may result in resistive heating at the target tissue (e.g., which may increase ablation potential). Moreover, the lattice structure of electrode 24 may increase the surface area of electrode 24, which may improve conductivity and/or dissipation of current. Furthermore, a more uniform power distribution may allow for more precise control of temperature, which may increase the safety of device 12. Additionally, the use of a coil electrode may result in a magnetic field when current is passed into the coil, which could lead to inductive current leakage. The use of latticed electrode 24 may reduce this inductive current leakage.

Electrode 24 may generally be formed from a suitable material including one or more of gold, a gold alloy, silver, a silver alloy, platinum, a platinum alloy, chromium, a chromium alloy, stainless steel, copper, a copper alloy, titanium, a titanium alloy, nickel, a nickel alloy, a nickel-titanium alloy, aluminum, and an aluminum alloy. These are just examples. Other materials are contemplated including those disclosed herein. Some of these and other materials may also provide a desirable level of radiopacity, which may aid in the visualization of device 12. It may be desirable to utilize materials that balance desirable electrical conductivity properties with physical properties (e.g., flexibility, trackability, and the like) and with material cost. In at least some embodiments, electrode 24 may include a shape memory material such as, for example, a nickel-titanium alloy. Such materials may allow electrode 24 (and/or device 12) to take a secondary shape (e.g., a curved or bent shape) in response to changes in temperature and/or exposure to current. These materials may also allow electrode 24 (and/or device 12) to return to the original shape (e.g., straight) upon removal of current.

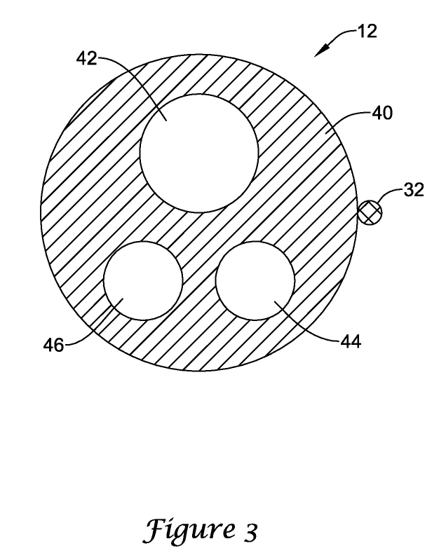

A cross-sectional view of shaft 40 of the renal nerve modulation device 12 proximal to balloon 22 is illustrated in FIG. 3. Shaft 40 may include a guidewire lumen 42, a lumen 44 connected to the fluid inlet 36, and a lumen 46 connected to the fluid outlet 38. Power wire 32 may extend along the outer surface of shaft 40 or may be embedded within the shaft. Power wire 32 proximal to the balloon may be electrically insulated and may be used to transmit power to the portion of the electrode 24 disposed within balloon 22. Other configurations are contemplated. In some embodiments, the guidewire lumen 42 and/or one of the fluid lumens 44/46 may be omitted. In some embodiments, guidewire lumen 42 may extend from the distal end of device 12 to a proximal hub. In other embodiments, the guidewire lumen can have a proximal opening that is distal the proximal portion of the system. In some embodiments, the fluid lumens 44/46 can be connected to a system to circulate the fluid through the balloon 22 or to a system that supplies new fluid and collects the evacuated fluid. It can be appreciated that embodiments may function with merely a single fluid lumen and a single fluid outlet into balloon 22.

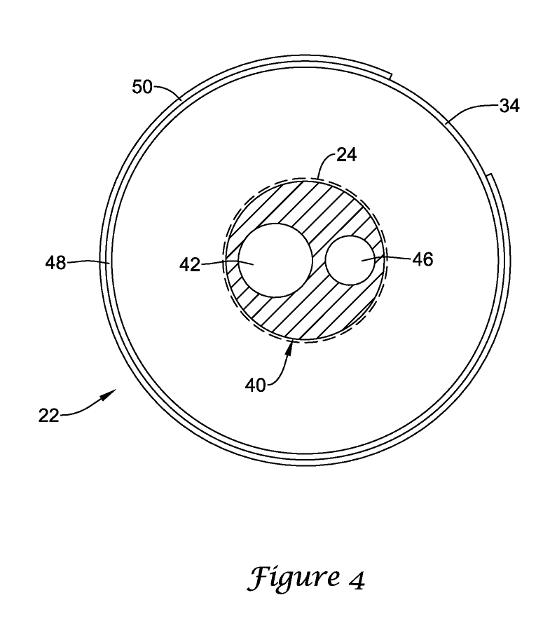

A cross-sectional view of the shaft 40 distal to fluid outlet 38 is illustrated in FIG. 4. The guidewire lumen 42 and the fluid inlet lumen 46 are present, as well as electrode 24 (represented in phantom line). In addition, balloon 22 is shown in cross-section as having a first layer 48 and a second layer 50. Window 34 may be formed in balloon 22 by the absence of second layer 50. First layer 48 may include an RF permeable material. One suitable material is a hydrophilic polyurethane. Other suitable materials include other hydrophilic polymers such as hydrophilic PEBAX, hydrophilic nylons, hydrophilic polyesters, block co-polymers with built-in hydrophilic blocks, polymers including ionic conductors, polymers including electrical conductors, metallic or nanoparticle filled polymers, and the like. Suitable hydrophilic polymers may exhibit between 20% to 50% hydrophilicity (or % water absorption). The second layer 50 may include an electrically non-conductive polymer such as a non-hydrophilic polyurethane, PEBAX, nylon, polyester, or block-copolymer. Other suitable materials include any of a range of electrically non-conductive polymers. The materials of the first layer and the second layer may be selected to have good bonding characteristics between the two layers. For example, a balloon 22 may be formed from a first layer 48 made from a hydrophilic PEBAX and a second layer 50 made from a regular or non-hydrophilic PEBAX. In other embodiments, a suitable tie layer (not illustrated) may be provided between the two layers.

In some of these and in other embodiments, a mask may be applied over hydrophilic material to reveal hydrophilic portions or windows 34. In an example, the mask can be a separate component into which balloon 22 is inserted. In another example, the mask may be applied onto the balloon 22. Some other details regarding masks and masking may be found in U.S. Pat. No. 7,736,362, the entire disclosure of which is herein incorporated by reference. Other details regarding masks and masking can be found appended at the end of this disclosure.

Prior to use, balloon 22 may be hydrated as part of the preparatory steps. Hydration may be effected by soaking the balloon in a saline solution. During ablation, a conductive fluid may be infused into balloon 22, for example via outlet 38. The conductive fluid may expand balloon 22 to the desired size. Balloon expansion may be monitored indirectly by monitoring the volume of conductive fluid introduced into the system or may be monitored through radiographic or other conventional means. Optionally, once balloon 22 is expanded to the desired size, fluid may be circulated within balloon 22 by continuing to introduced fluid through the fluid inlet 36 while withdrawing fluid from the balloon through the fluid outlet 38. The rate of circulation of the fluid may be between 2 and 20 ml/min, between 3 and 15 ml/min, between 5 and 10 ml/min or other desired rate of circulation. These are just examples. The circulation of the conductive fluid may mitigate the temperature rise of the tissue of a blood vessel in contact with the windows 34.

Electrode 24 may be activated by supplying energy to electrode 24. The energy may be supplied at 400-500 KHz at about 5-30 watts of power. These are just examples, other energies are contemplated. The energy may be transmitted through the medium of the conductive fluid and through windows 34 to the blood vessel wall to modulate or ablate the tissue. The second layer 50 of balloon 22 may prevent the energy transmission through the balloon wall except at windows 34 (which lack second layer 50). The progress of the treatment may be monitored by monitoring changes in impedance through electrode 24.

The electrode 24 may be activated for an effective length of time, such as 1 minute or 2 minutes. One the procedure is finished at a particular location, the balloon 22 may be partially or wholly deflated and moved to a different location such as the other renal artery, and the procedure may be repeated at another location as desired using conventional delivery and repositioning techniques.

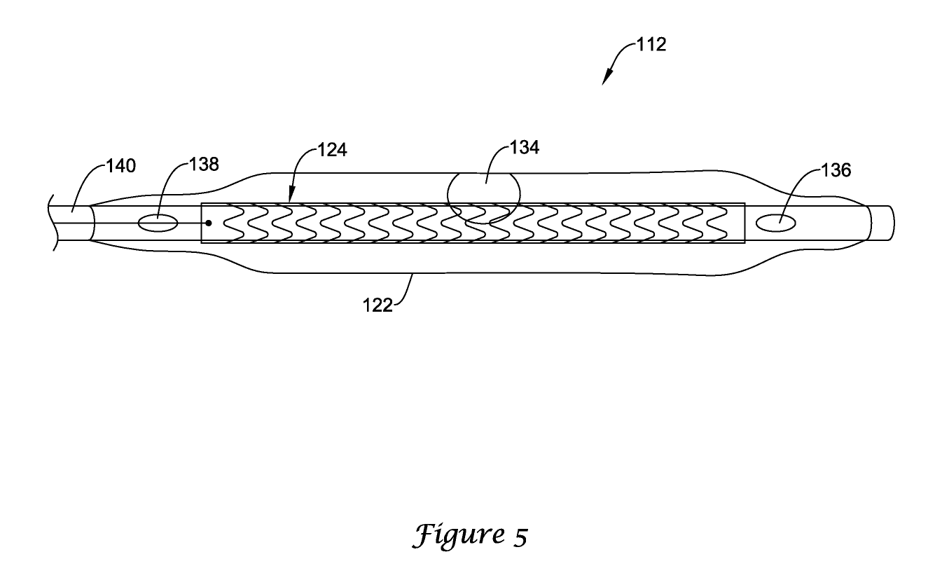

FIG. 5 illustrates another example renal nerve modulation device 112 that may be similar in form and function to other renal nerve modulation devices disclosed herein. Device 112 may include shaft 140 having balloon 122 coupled thereto. Shaft 140 may have ports 136/138 formed therein that may allow a conductive fluid to be circulated within balloon 122. Electrode 124 may be disposed within balloon 122 and may be used to supply energy to a single window or virtual electrode 134 formed on balloon 122.

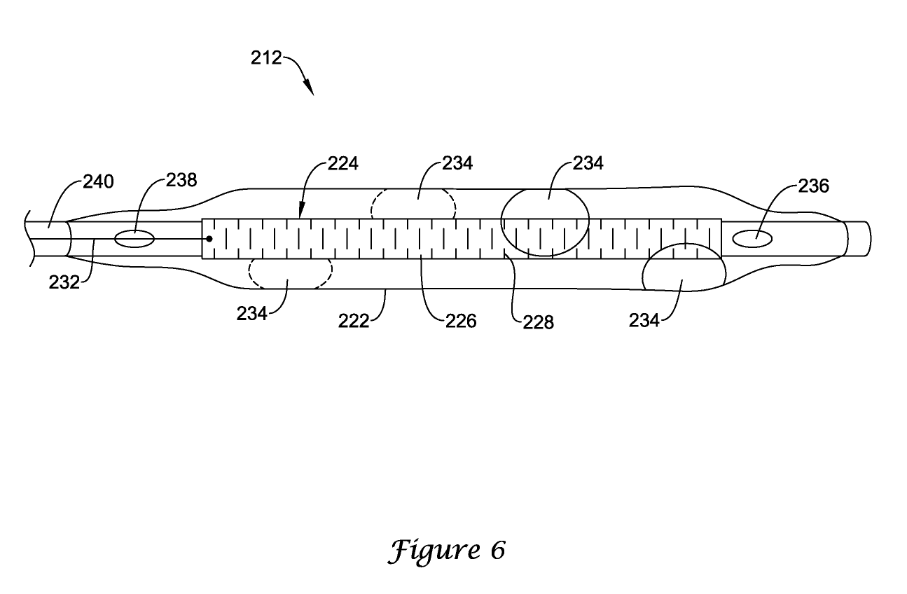

FIG. 6 illustrates another example renal nerve modulation device 212 that may be similar in form and function to other renal nerve modulation devices disclosed herein. Device 212 may include shaft 240 having balloon 222 coupled thereto. Shaft 240 may have ports 236/238 formed therein that may allow a conductive fluid to be circulated within balloon 222. One or more windows or virtual electrodes 234 may be formed on balloon 222.

Electrode 224 may take the form of a slotted tubular member including a tube body 226 having a plurality of slots 228 formed therein. A lead 232 may be attached to electrode 224. For example, electrode 224 may include a nickel-titanium alloy tubular member 226 having slots 228 formed therein. Such an electrode 224 may be suitably flexible and may provide uniform power distribution similar to other electrodes disclosed herein. A wide variety of patterns and/or configurations for slots 228 are contemplated. Some examples of the patterns and/or configurations for slots 228 are disclosed herein.

Forming electrode 224 may include a variety of processes. For example, electrode 224 may be formed from a flat stock of material that is cut using a suitable process (e.g., laser cut, cut via EDM, punch pressed, stamped, chemical etched, or the like) and then crimped, rolled, bent, and welded into a tubular form. Alternatively, electrode 224 may be formed from tube stock (and/or an extruded tube) that is cut using a suitable process. In still other embodiments, a metal sintering (powder metallurgy) process and/or a stereolithography process may be used to form electrode 224. To the extent applicable, similar processes may also be used to form electrode 24 and/or other electrodes disclosed herein.

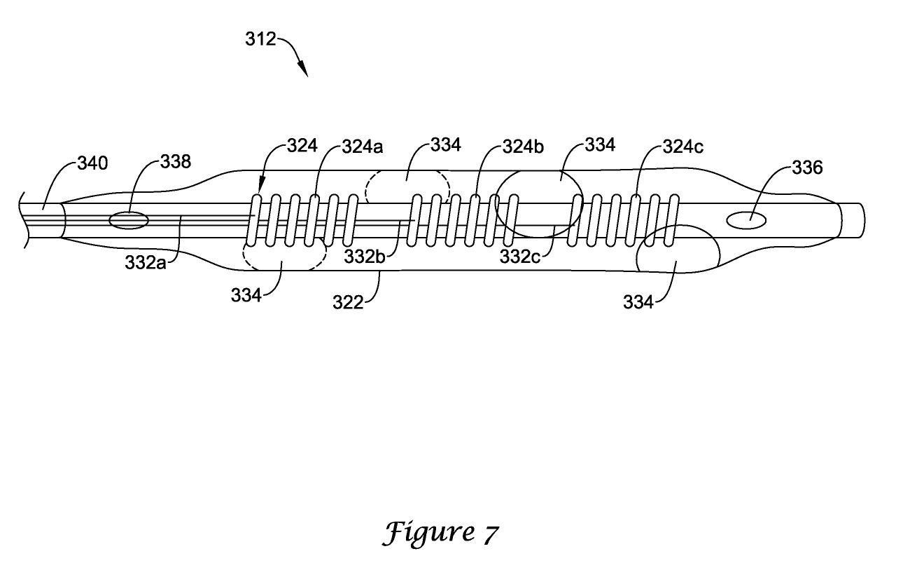

FIG. 7 illustrates another example renal nerve modulation device 312 that may be similar in form and function to other renal nerve modulation devices disclosed herein. Device 312 may include shaft 340 having balloon 322 coupled thereto. Shaft 340 may have ports 336/338 formed therein that may allow a conductive fluid to be circulated within balloon 322. One or more windows or virtual electrodes 334 may be formed on balloon 322.

Electrode 324 may take the form of a plurality of coil electrodes 324a/324b/324c disposed along shaft 340 and within balloon 322. Leads 332a/332b/332c may be attached to coil electrodes 324a/324b/324c, respectively. In at least some embodiments, coil electrodes 324a/324b/324c may be activated independently of one another. In this example, three coil electrodes 324a/324b/324c are utilized. This is not intended to be limiting, however, as any suitable number of coil electrodes may be utilized (e.g., one, two, three, four, five, six, seven, eight, or more). Coil electrodes 324a/324b/324c are also shown as being formed form a round wire wound about shaft 340 and having an open pitch. Other embodiments are contemplated where the shape of the coil wire is altered, the pitch of the coil is altered, etc. In addition, coil electrodes 324a/324b/324c are shown as being separate coils that are longitudinally spaced. Alternatively configurations are contemplated where coil electrodes 324a/324b/324c are interconnected or continuous with one another, overlap with one another, etc.

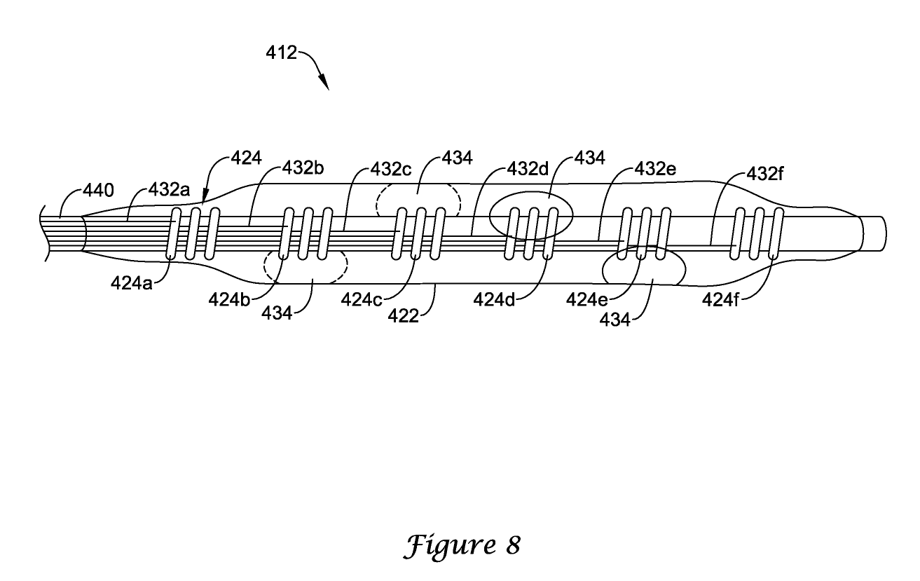

FIG. 8 illustrates another example renal nerve modulation device 412 that may be similar in form and function to other renal nerve modulation devices disclosed herein. Device 412 may include shaft 440 having balloon 422 coupled thereto. Electrode 424 may take the form of a plurality of coil electrodes 424a/424b/424c/424d/424e/424f disposed along shaft 440 and within balloon 422. Leads 432a/432b/432c/432d/432e/432f may be attached to coil electrodes 424a/424b/424c/424d/424e/424f, respectively. In at least some embodiments, coil electrodes 424a/424b/424c/424d/424e/424f may be activated independently of one another.

In this example, coil electrodes 424a/424b/424c/424d/424e/424f may be aligned with various structures of balloon 422. For example, coil electrodes 424a/424f are disposed adjacent to the proximal and distal waist of balloon 422. In at least some embodiments, coil electrodes 424a/424f may include a radiopaque material. Accordingly, coil electrodes 424a/424f may aid a clinician in visualization of balloon 422 during an intervention and/or aid in positioning device 412. In at least some embodiments, the use of radiopaque coil electrodes 424a/424f may allow device 412 to be manufactured without separate radiopaque markers that are typically used with balloons.

At least some of the other coil electrodes 424b/424c/424d/424e may be aligned with windows 434. This may allow for efficient transfer of energy from coil electrodes 424b/424c/424d/424e to windows 434. Coil electrodes 424b/424c/424d/424e may also include a radiopaque material, which may aid in visualization and/or positioning. In addition, the use of radiopaque coil electrodes 424b/424c/424d/424e aligned with windows 434 may allow the clinician to more accurately determine the position of the windows 434 (e.g., relative to the anatomy) during an intervention. It can be appreciated that the number of coil electrodes and windows may vary. In at least some embodiments, two coil electrodes may be utilized at or near the balloon cone portions (e.g., coil electrodes 424a/424f) and the remaining number of coil electrodes may be the same the number of balloon windows.

During an ablation procedure, it may be desirable to monitor one or more physical and/or biological parameter. For example, it may be desirable to monitor the temperature before, during, and after the procedure. In addition, it may also be desirable to monitor force (e.g., force, pressure, contact, and/or the like), impedance, nerve activity, blood flow, device orientation, hormones and/or other chemical or biochemical entities, pH levels, ultrasonic signals, and the like. Accordingly, any of the devices disclosed herein may include one or more sensors that may be utilized to sense a physical and/or biological parameter during an intervention.

The materials that can be used for the various components of renal nerve modulation device 12 (and/or other medical devices disclosed herein) may include those commonly associated with medical devices. For simplicity purposes, the following discussion makes reference to renal nerve modulation device 12. However, this is not intended to limit the devices and methods described herein, as the discussion may be applied to other similar medical devices disclosed herein.

Device 12 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of device 12 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of device 12 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of device 12 to achieve the same result.

In some embodiments, a degree of Magnetic Resonance Imaging (MRI) compatibility is imparted into device 12. For example, device 12 or portions thereof, may be made of a material that does not substantially distort the image and create substantial artifacts (i.e., gaps in the image). Certain ferromagnetic materials, for example, may not be suitable because they may create artifacts in an MRI image. Device 12 or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers for device 12 may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether)phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. Various embodiments of arrangements and configurations of slots are also contemplated that may be used in addition to what is described above or may be used in alternate embodiments. For simplicity purposes, the following disclosure makes reference to device 212, slots 228, and tubular member 226. However, it can be appreciated that these variations may also be utilized for other slots and/or tubular members. In some embodiments, at least some, if not all of slots 228 are disposed at the same or a similar angle with respect to the longitudinal axis of tubular member 226. As shown, slots 228 can be disposed at an angle that is perpendicular, or substantially perpendicular, and/or can be characterized as being disposed in a plane that is normal to the longitudinal axis of tubular member 226. However, in other embodiments, slots 228 can be disposed at an angle that is not perpendicular, and/or can be characterized as being disposed in a plane that is not normal to the longitudinal axis of tubular member 226.

Additionally, a group of one or more slots 228 may be disposed at different angles relative to another group of one or more slots 228. The distribution and/or configuration of slots 228 can also include, to the extent applicable, any of those disclosed in U.S. Pat. Publication No. US 2004/0181174, the entire disclosure of which is herein incorporated by reference.

Slots 228 may be provided to enhance the flexibility of tubular member 226 while still allowing for suitable torque transmission characteristics. Slots 228 may be formed such that one or more rings and/or tube segments interconnected by one or more segments and/or beams that are formed in tubular member 226, and such tube segments and beams may include portions of tubular member 226 that remain after slots 228 are formed in the body of tubular member 226. Such an interconnected structure may act to maintain a relatively high degree of torsional stiffness, while maintaining a desired level of lateral flexibility. In some embodiments, some adjacent slots 228 can be formed such that they include portions that overlap with each other about the circumference of tubular member 226. In other embodiments, some adjacent slots 228 can be disposed such that they do not necessarily overlap with each other, but are disposed in a pattern that provides the desired degree of lateral flexibility.

Additionally, slots 228 can be arranged along the length of, or about the circumference of, tubular member 226 to achieve desired properties. For example, adjacent slots 228, or groups of slots 228, can be arranged in a symmetrical pattern, such as being disposed essentially equally on opposite sides about the circumference of tubular member 226, or can be rotated by an angle relative to each other about the axis of tubular member 226. Additionally, adjacent slots 228, or groups of slots 228, may be equally spaced along the length of tubular member 226, or can be arranged in an increasing or decreasing density pattern, or can be arranged in a non-symmetric or irregular pattern. Other characteristics, such as slot size, slot shape, and/or slot angle with respect to the longitudinal axis of tubular member 226, can also be varied along the length of tubular member 226 in order to vary the flexibility or other properties. In other embodiments, moreover, it is contemplated that the portions of the tubular member, such as a proximal section, or a distal section, or the entire tubular member 226, may not include any such slots 228.

As suggested herein, slots 228 may be formed in groups of two, three, four, five, or more slots 228, which may be located at substantially the same location along the axis of tubular member 226. Alternatively, a single slot 228 may be disposed at some or all of these locations. Within the groups of slots 228, there may be included slots 228 that are equal in size (i.e., span the same circumferential distance around tubular member 226).

In some of these as well as other embodiments, at least some slots 228 in a group are unequal in size (i.e., span a different circumferential distance around tubular member 226). Longitudinally adjacent groups of slots 228 may have the same or different configurations. For example, some embodiments of tubular member 226 include slots 228 that are equal in size in a first group and then unequally sized in an adjacent group. It can be appreciated that in groups that have two slots 228 that are equal in size and are symmetrically disposed around the tube circumference, the centroid of the pair of beams (i.e., the portion of tubular member 226 remaining after slots 228 are formed therein) is coincident with the central axis of tubular member 226. Conversely, in groups that have two slots 228 that are unequal in size and whose centroids are directly opposed on the tube circumference, the centroid of the pair of beams can be offset from the central axis of tubular member 226. Some embodiments of tubular member 226 include only slot groups with centroids that are coincident with the central axis of the tubular member 226, only slot groups with centroids that are offset from the central axis of tubular member 226, or slot groups with centroids that are coincident with the central axis of tubular member 226 in a first group and offset from the central axis of tubular member 226 in another group. The amount of offset may vary depending on the depth (or length) of slots 228 and can include other suitable distances.

Slots 228 can be formed by methods such as micro-machining, saw-cutting (e.g., using a diamond grit embedded semiconductor dicing blade), electron discharge machining, grinding, milling, casting, molding, chemically etching or treating, or other known methods, and the like. In some such embodiments, the structure of the tubular member 226 is formed by cutting and/or removing portions of the tube to form slots 228. Some example embodiments of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing device 212 may include forming slots 228 tubular member 226 using these or other manufacturing steps.

In at least some embodiments, slots 228 may be formed in tubular member using a laser cutting process. The laser cutting process may include a suitable laser and/or laser cutting apparatus. For example, the laser cutting process may utilize a fiber laser. Utilizing processes like laser cutting may be desirable for a number of reasons. For example, laser cutting processes may allow tubular member 226 to be cut into a number of different cutting patterns in a precisely controlled manner. This may include variations in the slot width, ring width, beam height and/or width, etc. Furthermore, changes to the cutting pattern can be made without the need to replace the cutting instrument (e.g., blade). This may also allow smaller tubes (e.g., having a smaller outer diameter) to be used to form tubular member 226 without being limited by a minimum cutting blade size. Consequently, tubular member 226 may be fabricated for use in neurological devices or other devices where a relatively small size may be desired. A laser cutting process may also be utilized to form latticed electrode 24.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A renal nerve modulation device, comprising:
an elongate shaft;
a balloon coupled to the shaft, the balloon having a hydrophilic electrode region; and
a flexible electrode coupled to the shaft and disposed within the balloon, the flexible electrode having a plurality of openings formed therein.

2. The renal nerve modulation device of claim 1, wherein the balloon includes an inner layer and an outer layer.

3. The renal nerve modulation device of claim 2, wherein the hydrophilic electrode region is defined by the absence of the outer layer along a portion of the balloon.

4. The renal nerve modulation device of claim 1, wherein the balloon includes one or more additional hydrophilic electrode regions.

5. The renal nerve modulation device of claim 1, wherein a conductive fluid is disposed within the balloon.

6. The renal nerve modulation device of claim 1, wherein the flexible electrode is a radiofrequency electrode.

7. The renal nerve modulation device of claim 1, wherein the flexible electrode includes a stent-like lattice.

8. The renal nerve modulation device of claim 1, wherein the flexible electrode includes a tubular member having a plurality of slots formed therein.

9. The renal nerve modulation device of claim 1, wherein the flexible electrode includes a nickel-titanium alloy.

10. The renal nerve modulation device of claim 1, wherein the flexible electrode includes one or more of gold, a gold alloy, silver, a silver alloy, platinum, a platinum alloy, chromium, a chromium alloy, stainless steel, copper, a copper alloy, titanium, a titanium alloy, aluminum, and an aluminum alloy.

11. The renal nerve modulation device of claim 1, wherein a power wire is attached to the flexible electrode and extends proximally therefrom.

12. A renal nerve modulation device, comprising:
an elongate shaft;
a balloon coupled to the shaft, the balloon including an inner layer and an outer layer;
wherein a hydrophilic electrode region is defined in the balloon by the absence of the outer layer along a portion of the balloon; and
a tubular electrode disposed about the shaft and having a plurality of openings formed therein.

13. The renal nerve modulation device of claim 12, wherein the balloon includes one or more additional hydrophilic electrode regions.

14. The renal nerve modulation device of claim 12, wherein a conductive fluid is disposed within the balloon.

15. The renal nerve modulation device of claim 12, wherein the tubular electrode is a radiofrequency electrode.

16. The renal nerve modulation device of claim 12, wherein the tubular electrode includes a stent-like lattice.

17. The renal nerve modulation device of claim 16, wherein the stent-like lattice has an end pad coupled thereto and wherein a power wire is attached to the end pad and extends proximally therefrom.

18. The renal nerve modulation device of claim 12, wherein the tubular electrode includes a nickel-titanium alloy.

19. The renal nerve modulation device of claim 12, wherein the renal nerve modulation device is free of a coil electrode.

20. A renal nerve modulation device, comprising:
an elongate shaft;
a balloon coupled to the shaft, the balloon having a hydrophilic electrode region; and
an electrode coupled to the shaft and disposed within the balloon, the electrode including a plurality of coils.
